(19) United States
(12) Patent Application Publication
(10) Pub. No.: US 2008/0193372 A1
(43) Pub. Date: Aug. 14, 2008

(54) Efficacy and Safety of Targeted Particulate Agents with Decoy Systems

(75) Inventors: Gregory M. Lanza, St. Louis, MO (US); Samuel A. Wickline, St. Louis, MO (US)

Correspondence Address: MORRISON & FOERSTER LLP, 12531 HIGH BLUFF DRIVE, SUITE 100, SAN DIEGO, CA, 92130-2040, US

(73) Assignee: BARNES-JEWISH HOSPITAL, St. Louis, MO (US)

(21) Appl. No.: 10/588,572

(22) PCT Filed: Feb. 10, 2005
(86) PCT No.: PCT/US05/04858

§ 371 Date: Nov. 30, 2007

Related U.S. Application Data
(60) Provisional application No. 60/543,761, filed on Feb. 10, 2004.

(51) Int. Cl. A61K 51/00; A61K 9/127; A61K 39/00; A61K 49/06; A61K 49/22; A61K 49/04; A61K 47/00

(52) U.S. Cl. 424/1.11; 424/450; 424/130.1; 514/772; 424/9.3; 424/9.4; 424/9.5

(57) ABSTRACT

A decoy inactive carrier composition is administered simultaneously with a targeted composition containing vehicles for delivering a desired agent to a biological target. This simultaneous administration enhances the delivery of the targeted composition to the desired location in a subject.

### CROSS-REFERENCE TO RELATED APPLICATION

This application claims benefit under 35 U.S.C. §119(e) to U.S. provisional application Serial No. 60/543,761 filed 10 Feb. 2004. The contents of this document are incorporated herein by reference.

### STATEMENT OF RIGHTS TO INVENTIONS MADE UNDER FEDERALLY SPONSORED RESEARCH

This invention was made, in part, with U.S. government support. The U.S. government has certain rights in this invention.

### TECHNICAL FIELD

The invention relates to methods to deliver targeted agents for ultrasound, X-ray, radioimaging, MRI, and/or therapeutic uses. In particular, it relates to methods to improve or maintain effectiveness of targeted agents while reducing the total dosage of active particles by concomitant administration of “decoy” delivery vehicles.

### BACKGROUND ART

Successful and specific targeting of diagnostic and therapeutic agents to desired locations in a subject has been problematic. Solutions to this problem where the targeting conjugate is non-particulate has involved the use of clearing agents. One approach, where an agent to be delivered to a target site is rapidly cleared by the kidneys due to its small size is described in U.S. Pat. No. 4,863,713. In this instance, the target site is first provided with a cognate for a binding ligand conjugated to the agent to be delivered and the cognate is allowed to localize over time at the target site. Circulating excess cognate is then removed by administering a clearing agent that reacts with the circulating binding protein to form an aggregate that can be cleared rapidly by the reticuloendothelial system (RES) which is primarily operated by the liver and spleen. The small molecule comprising the targeting ligand and agent to be delivered is then administered and the unbound administered conjugate is rapidly cleared by the kidneys while the cognate residing at the target site captures some of it. Particular components useful in this approach are described in U.S. Pat. No. 5,616,690. This may or may not be entirely successful. Problems remain when the substance to be targeted to a particular tissue or location in a subject is of sufficient size to be cleared by the reticuloendothelial system (RES).

The systemic clearance of particles (from nano to micro scale) by the RES, in particular, liver and spleen, has minimized the effectiveness of site-specific, i.e., targeted imaging and/or therapeutic agents. Typically, a third or more of the total dose of these agents administered systemically is cleared by the liver and spleen with no therapeutic benefit and potential adverse clinical sequelae. Many attempts have been made to delay the rapid clearance of particulate agents by these RES organs, most notably the utilization of polyethylene glycol surface coatings, which extend the circulating half-life of many particulate systems. These agents are often referred to as being “stealthy.”

Other approaches have included two-step efforts to saturate the RES capacity to clear particulates with unlabeled carrier or other related materials followed later by administering a active imaging and/or therapeutic agent. For example, in attempting to label tumors with radionuclides, the radionuclides have been coupled to antibodies or fragments that bind to tumor associated antigens. However, in order to avoid massive doses, subjects have first been administered unlabeled antibodies which then, presumably, saturate the liver and spleen, permitting the labeled antibodies to progress without dilution by the RES to the target area. This method has also been applied to particulate delivery systems, especially in the case of relatively small particles—i.e., on the order of 100-200 nm, which have very long half-lives. This strategy presents serious flaws. The unlabeled antibodies, fragments or peptides are typically unable to saturate the natural clearance mechanisms and they can compete and blockade the targeted sites without contributing to imaging and/or therapeutic efficacy.

It appears that the RES is not readily saturated without inducing symptoms associated with liver or splenic congestion, including discomfort, nausea or vomiting. Thus, the potential decrease in efficacy in combination with adverse clinical effects appears to account for the failure of the previous approaches. The present invention solves this problem by employing a probability-based approach—i.e., a non-targeted agent of similar physical character is co-administered, i.e., simultaneously, with targeted agent, to facilitate the evasion of the RES system by the targeted complex, which provides improved uptake at the desired site. Since the dosage of agent required for efficacy at the targeted site is small in comparison to the amount cleared by the RES system, the use of decoys often allows the total dose of active agent to be lower than what would otherwise be required to compensate for RES losses. This translates directly into lower exposure of patients to active agents (i.e., drugs, metals, delivery vehicle components, etc.) and lowers the per dosage cost of the agent (i.e., giving less costs less). Lowering the costs and improving the efficacy of targeted agents is necessary to make some otherwise costly agents viable in clinical medicine.

### DISCLOSURE OF THE INVENTION

The invention is directed to an improvement in methods for supplying contrast media or labeling or therapeutic agents to desired targets at reduced dosages and resulting in equivalent enhanced effectiveness with improved safety and health benefits. The method comprises administering, simultaneously, an image contrast or image generating agent and/or a therapeutic agent coupled to a nanoparticulate carrier, which is itself coupled to a targeting agent specific for the desired target or a complementary ligand, in the presence of an excess of untargeted carrier. The carrier for the desired agent need not be identical to the “decoy” carrier used as a diluent to swamp the RES, however, the decoy must mimic the behavior of the labeled, targeted carrier.

As the potency of the imaging and or therapeutic agent increases, the ratio of excess decoy agents to targeted agents may increase. Thus, for example, nuclear agents that provide signal sensitivity may be administered with a great excess of decoy whereas the dosage of MRI or CT agents, which provide less detectable signal per unit of agent may be administered with only modest amounts of decoy, e.g., 2-10 fold the amount of active composition. Similarly, therapeutic agents with very high potency may be mixed with decoys at very high ratios of decoy to active agent and maintain therapy. In short, as the potency of the imaging agent and or therapeutic agent increases, the need to saturate the target with the agent for maximum effect declines and conversely, weaker agents must be provided at higher dosages to ensure adequate effectiveness.

Thus, in one aspect, the invention is directed to a method to provide a contrast agent, labeling agent and/or a therapeutic agent to a target location in a subject which method comprises administering substantially simultaneously to said subject:

(1) an active composition of targeted particulate vehicles, said vehicles coupled to a binding moiety which binds specifically to a cognate at the target site and optionally comprising therapeutic agent and/or labeling component which is a contrast agent or signal-generating agent; and

(2) a composition comprising decoy particulate vehicles lacking said specific binding moiety and optionally lacking said labeling component and/or therapeutic agent;

wherein the ratio of the vehicles of (2) to the vehicles of (1) is sufficient to increase the concentration of targeted vehicles at the desired site and/or to reduce the effective dosage of targeted vehicles required.

In still another aspect, the invention is directed to compositions which contain mixtures of the targeted vehicles and decoy vehicles of items (1) and (2) above in the desired ratio of decoy:targeted vehicles comprising targeting agent.

### BRIEF DESCRIPTION OF THE DRAWINGS

FIG. 1 shows the contrast obtained in radionuclide imaging when targeted nanoparticles containing ¹¹¹In are administered alone and when administered with decoy inactive carrier, said administration being simultaneous.

FIG. 2 shows results of an experiment similar to FIG. 1 when the inactive carrier decoy is administered well prior to the targeted active composition.

FIG. 3 shows the results of radionuclide imaging using nanoparticles containing varying amounts of radionuclide per particle.

FIG. 4 shows imaging results with labeled nanoparticles in the presence of varying amounts of decoy inactive carrier.

### MODES OF CARRYING OUT THE INVENTION

The invention relies on the presence of a large excess of untargeted “carrier” or “decoy” vehicles to compete with targeted vehicles for uptake by the reticuloendothelial system (RES) when supplied in the presence of vehicles that are targeted to a desired location.

The RES can remove certain amounts of particles from the system each pass, and the decoys prevent 100% of that capacity being effective to remove targeted particles. The half-life of particles subject to the RES is dose dependent, i.e., the circulating half-life of particles increases as the dosage increases. The slower clearance for higher dosages is used to advantage in the invention method by maintaining a high dosage of total particles while decreasing the number of targeted ones. Thus, the increased half-life for all particles due to the higher dosage benefits targeted particles. A biexponential model and focus on the beta elimination rate for a given dose can be used to model this behavior.

The invention relies on concurrent administration of untargeted “carrier” or “decoy” vehicles to reduce the premature RES clearance of targeted vehicles. Depending on the potency of the targeted vehicles, decoys may represent between 50% to greater than 99.999% of the administered dosage.

By use of the method of the invention, it is possible to diminish the dosage of the vehicles that carry the targeting moiety and possible additional “activity components.” In general, the greater the ratio of decoy inactive carrier to the composition with active vehicles, the more effective the imaging or treatment becomes at lower dosages of the active composition. In addition, the half-life of the particles in the active composition may be enhanced by structural changes in the particles—e.g., PEGylation, decrease in diameter, and surfactant changes. An upper limit to the decoy approach of the invention resides in the limitation of volume of compositions that can be effectively administered to a subject.

Using the invention method, it has been possible to achieve successful targeting in the range of 10% of the labeled targeted composition whereas without the method of the invention, only 2% or more typically only 1% or less of the targeted particles actually reach the target.

As used herein, “vehicles” refers to nanoparticulates or microparticulates that perform the desired drug delivery or imaging function or generally, particles cleared by the RES. The vehicles may, for example, be liposomes, nanoparticles, micelles, lipoproteins, immunoconjugate dendrimers, hydrogels, polymeric systems and the like. They may also be bubbles containing gas and/or gas precursors, particulates comprising hydrocarbons and/or halocarbons, hollow or porous particles or solids. In general, the particulates may be solid particulates which may be coated with additional material, may be liquid cores surrounded by solid or liquid outer layers, or may contain gas or gas precursors again surrounded by solid or liquid outer layers. The vehicles may be supplied in the form of emulsions. The particulate vehicles in the active compositions are coupled to targeting moieties that selectively bind to a desired tissue or location in a subject. The targeting moiety may be a ligand specific for a cognate that resides naturally on the targeted tissue or may be the cognate of an artificially supplied moiety, for example, avidin which will bind to a biotin-labeled targeted tissue.

These targeting moieties may be antibodies or fragments thereof, peptidomimetics, small molecule ligands, aptamers and the like. They are coupled, either covalently or non-covalently, to the vehicles in the active composition.

Depending on the function of the active composition, further components may be present in the vehicles, or further components may not be necessary. For example, if the vehicles are to be concentrated at a tissue site for ultrasound imaging, the use of some particles, such as, e.g., perfluorocarbon nanoparticles, may supply sufficient contrast without further modification. Similarly, if the vehicles are opaque to X-rays, no further activity component may be needed. This may suffice as well for MRI imaging due to the presence of ¹⁹F nuclei in the particles. However, it may also be desired to image the desired location using radioimaging, or MRI based on proton spin may be desirable. In this instance, the vehicles may further be coupled to radionuclide or to chelating agents either directly or through linkers, which chelators may in turn include heavy metal ions or radionuclides. Similarly, if the purpose of targeting the vehicles is to deliver drugs, these vehicles will also include a therapeutically active agent. In general, the “active composition” comprises particulate vehicles coupled to a moiety that targets a desired location in a subject through specific binding thereto, and will further comprise, as consistent with the purpose of the targeting, a labeling agent, a contrast agent, a therapeutically active agent, or other component whose delivery to a selective location is desired, which agents or “activity components” may be inherent to the vehicles.

The “active composition” is administered substantially simultaneously with a large excess of “inactive carrier.” The “inactive carrier” is a non-targeted composition which may comprise particulates similar to the vehicles in the active composition or particulates which are dissimilar in structure but mimic the behavior of the active composition. For example, the inactive carrier may simply comprise an emulsion where the particulates are oil droplets, such as the commercially available Intralipid®. The decoys in the “inactive carrier” or “non-targeted carrier” could be polymers, hydrogels, and the like, that are structurally dissimilar from the vehicles in the active composition, so long as they clear via the same mechanism, so as to compete for clearance by the RES. The particulates in the inactive composition do not contain targeting agent, and optionally do not contain the labeling agent, contrast agent or therapeutic agent characteristic of the active composition. Preferably, the particulates in the non-targeted carrier do not contain labeling/therapeutic agent, i.e., “activity component.”

For convenience, the term “activity component” has been coined to refer to whatever property is associated with the targeted vehicles that provides the desired result The activity component may be a moiety coupled to the vehicle—e.g., a radionuclide, a drug, a biological agent, a chelate containing a heavy metal, and the like, which coupling may be covalent or non-covalent. However, the “activity component” may be inherent to the vehicle itself—such as the use of perfluorocarbon-based or gas-bubble based particles as ultrasound contrast agents or the use of fluorinated hydrocarbon particles in ¹⁹F based MRI. Depending on the nature of the “activity component,” it may or may not be present in the non-targeted carrier.

Thus, both the active composition and the inactive carrier comprise particulates where the essential difference between said particulates is that those in the active composition contain a targeting moiety whereas those in the inactive carrier do not. “Activity component” may be present in both compositions, or only in the active composition. In some cases, the “activity component” is inherent in the vehicles themselves.

As stated above, the vehicles themselves may be of various physical states, including solid particles, solid particles coated with liquid, liquid particles coated with liquid, and gas particles coated with solid or liquid. Various vehicles useful in the invention have been described in the art as well as means for coupling targeting components to those vehicles in the active composition. Such vehicles are described, for example, in U.S. Pat. Nos. 6,548,046; 6,821,506; 5,149,319; 5,542,935; 5,585,112; 5,149,319; 5,922,304; and European publication 727,225, all incorporated herein by reference with respect to the structure of the vehicles. These documents are merely exemplary and not all-inclusive of the various kinds of particulate vehicles that are useful in the invention. The vehicles useful in the invention are specifically defined as those that are particles cleared by the RES.

The design of the inactive carrier as compared to the active composition will follow standard rational design principles—e.g., if the active composition is designed for targeted drug delivery, the inactive carrier will not contain the targeting agent and optionally not contain the drug to be delivered. If the active composition is designed for ultrasound imaging and comprises perfluorocarbon particles, the inactive carrier will comprise particulates that are preferably comparatively transparent to ultrasound imaging, such as oil droplets. If the active composition is designed for radiolabeling, the inactive carrier will preferably lack radionuclides, at least radionuclides of the type designed for labeling the target. If the active composition is directed to improving magnetic resonance imaging, the active composition may contain a chelate of a heavy metal ion whereas the inactive carrier may lack chelating agents. Thus, the inactive carrier is “inert” in the sense that it (a) fails to selectively bind the target tissue and (b) does not substantially interfere with or mimic the signal or biological activity of the active composition.

Typically, the ratio of inactive carrier to active composition is in the range of 1:1 to 10⁵:1. Intermediate ratios may also be used, on a scale dependent on the nature of the active composition. Thus, ratios of 2:1, 5:1, 10:1, 50:1, 100:1, 10³:1, 10⁴:1, 10⁵:1 and values between these are all within the scope of the invention. As noted above, the more potent the activity component, the greater the ratio may be, as upper dosage limits for both active composition and decoy are not exceeded. For radionuclides, typical ratios are in the higher ranges, while for MRI or CT agents the amount of targeted material that must remain at the target site is greater so that less decoy can be tolerated while maintaining a dosage within practical levels. The ratio is determined as the number of particulates administered. This ratio is readily determined when similar particulates are used in both inactive carrier and active composition; more sophisticated methods are required when, for example, the active composition utilizes perfluorocarbon nanoparticles while the inactive carrier is an Intralipid® emulsion. However, methods for measuring particulate concentrations are well known in the art.

By “substantially simultaneous” administration is meant that active composition and the inactive carrier are administered so that their initial bio-distribution will be co-extensive,—i.e., the active composition and the inactive carrier are administered within five minutes of each other, preferably within two minutes of each other, preferably within one minute of each other, preferably within 30 seconds of each other and preferably exactly simultaneously as a single composition or concomitant administration of two compositions. If there is any interval at all, the inactive carrier is preferably administered first.

The “vehicles” that are contained in either the active composition or the inactive carrier may be microparticulate or nanoparticulate agents, i.e., particulates cleared by the RES. The vehicles are considered “particulate” if they are cleared by the RES. As stated above, included in such particulates are nanoparticles with lipophilic cores optionally coated with a lipid/surfactant coating that is useful in anchoring desired moieties, such as chelating agents, targeting agents, and the like to the particles.

The inert core can be a vegetable, animal or mineral oil, or fluorocarbon compound—perfluorinated or otherwise rendered additionally inert. Mineral oils include petroleum derived oils such as paraffin oil and the like. Vegetable oils include, for example, linseed, safflower, soybean, castor, cottonseed, palm and coconut oils. Animal oils include tallow, lard, fish oils, and the like. Many oils are triglycerides.

Fluorinated liquids are included as cores. These include straight chain, branched chain, and cyclic hydrocarbons, preferably perfluorinated. Some satisfactorily fluorinated, preferably perfluorinated organic compounds useful in the particles of the invention themselves contain functional groups. Perfluorinated hydrocarbons are preferred. The nanoparticle core may comprise a mixture of such fluorinated materials. Typically, at least 50% fluorination is desirable in these inert supports. Preferably, the inert core has a boiling point of above 20° C., more preferably above 30° C., still more preferably above 50° C., and still more preferably above about 90° C.

Thus, the perfluoro compounds that are particularly useful in the above-described nanoparticle aspect of the invention include partially or substantially or completely fluorinated compounds. Chlorinated, brominated or iodinated forms may also be used.

With respect to any coating on the nanoparticles, a relatively inert core is provided with a lipid/surfactant coating that will serve to anchor the desired moieties to the nanoparticle itself. If an emulsion is to be formed, the coating typically should include a surfactant. Typically, the coating will contain lecithin type compounds which contain both polar and non-polar portions as well as additional agents such as cholesterol. Typical materials for inclusion in the coating include lipid surfactants such as natural or synthetic phospholipids, but also fatty acids, cholesterols, lysolipids, sphingomyelins, tocopherols, glucolipids, stearylamines, cardiolipins, a lipid with ether or ester linked fatty acids, polymerized lipids, and lipid conjugated polyethylene glycol. Other surfactants are commercially available.

The foregoing may be mixed with anionic and cationic surfactants.

Fluorochemical surfactants may also be used. These include perfluorinated alcohol phosphate esters and their salts; perfluorinated sulfonamide alcohol phosphate esters and their salts; perfluorinated alkyl sulfonamide alkylene quaternary ammonium salts; N,N-(carboxyl-substituted lower alkyl) perfluorinated alkyl sulfonamides; and mixtures thereof. As used with regard to such surfactants, the term “perfluorinated” means that the surfactant contains at least one perfluorinated alkyl group.

Typically, the lipids/surfactants are used in a total amount of 0.01-5% by weight of the nanoparticles, preferably 0.1-2% by weight. In one embodiment, lipid/surfactant encapsulated emulsions can be formulated with cationic lipids in the surfactant layer that facilitate the adhesion of nucleic acid material to particle surfaces. Cationic lipids include DOTMA, N-[1-(2,3-dioleoyloxy)propyl]-N,N,N-trimethylammonium chloride; DOTAP, 1,2-dioleoyloxy-3-(trimethylammonio)propane; and DOTB, 1,2-dioleoyl-3-(4′-trimethyl-ammonio) butanoyl-sn-glycerol may be used. In general the molar ratio of cationic lipid to non-cationic lipid in the lipid/surfactant monolayer may be, for example, 1:1000 to 2:1, preferably, between 2:1 to 1:10, more preferably in the range between 1:1 to 1:2.5 and most preferably 1:1 (ratio of mole amount cationic lipid to mole amount non-cationic lipid, e.g., DPPC). A wide variety of lipids may comprise the non-cationic lipid component of the emulsion surfactant, particularly dipalmitoylphosphatidylcholine, dipalmitoylphosphatidyl-ethanolamine or dioleoylphosphatidylethanolamine in addition to those previously described. In lieu of cationic lipids as described above, lipids bearing cationic polymers such as polyamines, e.g., spermine or polylysine or polyarginine may also be included in the lipid surfactant and afford binding of a negatively charged therapeutic, such as genetic material or analogues there of, to the outside of the emulsion particles.

Other particulate supports may also be used in carrying out the method of the invention. For example, the particles may be liposomal particles, or lipoproteins such as HDL, LDL and VLDL. The literature describing various types of liposomes is vast and well known to practitioners. In general, liposomes are comprised of one or more amphiphilic moieties and a steroid, such as cholesterol. They may be unilamellar, multilamellar, and come in various sizes. These lipophilic features can be used to couple to the chelating agent in a manner similar to that described above with respect to the coating on the nanoparticles having an inert core; alternatively, covalent attachment to a component of the liposomes can be used. Micelles are composed of similar materials, and this approach to coupling desired materials, and in particular, the chelating agents applies to them as well. Solid forms of lipids may also be used.

In addition, proteins or other polymers can be used to form the particulate carrier. These materials can form an inert core to which a lipophilic coating is applied, or the chelating agent can be coupled directly to the polymeric material through techniques employed, for example, in binding affinity reagents to particulate solid supports. Thus, for example, particles formed from proteins can be coupled to tether molecules containing carboxylic acid and/or amino groups through dehydration reactions mediated, for example, by carbodiimides. Sulfur-containing proteins can be coupled through maleimide linkages to other organic molecules which contain tethers to which the chelating agent is bound. Depending on the nature of the particulate carrier, the method of coupling so that an offset is obtained between the dentate portion of the chelating agent and the surface of the particle will be apparent to the ordinarily skilled practitioner.

Further, the particles used as vehicles may contain bubbles of gas or precursors which form bubbles of gas when in use. In these cases, the gas is contained in a liquid or solid based coating.

Other suitable vehicles which may be provided with targeting agents and optionally activity components or used in the carrier include the oil and water emulsions described in U.S. Pat. No. 5,536,489, liposome compositions such as those described in U.S. Pat No. 5,512,294 and oil and water emulsions as described in U.S. Pat. No. 5,171,737.

Thus, the vehicles used in the invention methods may be of a wide variety; the requirement for the method of the invention is that the vehicles present in the active composition behave, with respect to the RES, in a manner similar to the vehicles present in the inactive carrier. More than one type of vehicle may be used in either the active composition or in the inactive carrier or both. In the present discussion, vehicles “of the same composition” is defined to mean vehicles whose basic construction is the same although complementation with a targeting ligand and/or activity component may vary. Thus, “vehicles of the same composition” may be liposomes that are similarly constructed although one set of such liposomes, such as those used in the active composition, will further contain a targeting ligand and optionally an activity component. Vehicles “of the same composition” would include, for another example, microbubbles of the same gas and general dimensions although some contain targeting ligands and others do not. Conversely, vehicles “not of the same composition” are the basic particles of different construction, such as oil droplets versus fluorocarbon nanoparticles or gas microbubbles encapsulated with liquid as opposed to gas microbubbles encapsulated with a solid component. The requirement with regard to the method of the invention is that the active composition and the inactive carrier comprise vehicles that behave similarly with regard to their clearance characteristics in the RES, regardless of whether they are of the “same composition.”

The vehicles in the active composition comprise a targeting agent—i.e., a moiety which binds specifically to a cognate in the target tissue. Typically, the inactive carrier vehicles do not contain any targeting agent; it is theoretically possible that they could contain a targeting agent for a different target than that represented by the active composition; there would be no particular point, however, in doing this. In any event, they lack any moiety that binds to the target of interest. The choice of targeting agent will depend on the nature of the organ or tissue or type of tissue to be targeted. Typical targets include fibrin clots, liver, pancreas, neurons, tumor tissue, i.e., any tissue characterized by particular cell surface or other ligand-binding moieties. In order to effect this targeting, a suitable ligand is coupled to the particle directly or indirectly. An indirect method is described in U.S. Pat. No. 5,690,907, incorporated herein by reference. In this method, the lipid/surfactant layer of a nanoparticle is biotinylated and the targeted tissue is coupled to a biotinylated form of a ligand that binds the target specifically. The biotinylated nanoparticle then reaches its target through the mediation of avidin which couples the two biotinylated components.

Alternatively, the specific ligand itself is coupled directly to the particle, preferably but not necessarily, covalently. Thus, in such “direct” coupling, a ligand which is a specific binding partner for a target contained in the desired location is itself linked to the components of the particle, as opposed to indirect coupling where a biotinylated ligand resides at the intended target. Such direct coupling can be effected through linking molecules or by direct interaction with a surface component. Homobifunctional and heterobifunctional linking molecules are commercially available, and functional groups contained on the ligand can be used to effect covalent linkage. Typical functional groups that may be present on targeting ligands include amino groups, carboxyl groups and sulfhydryl groups. In addition, crosslinking methods, such as those mediated by glutaraldehyde could be employed. For example, sulfhydryl groups can be coupled through an unsaturated portion of a linking molecule or of a surface component; amides can be formed between an amino group on the ligand and a carboxyl group contained at the surface or vice versa through treatment with dehydrating agents such as carbodiimides. A wide variety of methods for direct coupling of ligands to components of particles in general and to components such as those found in a lipid/surfactant coating in one embodiment are known in the art.

In slightly more detail, for coupling by covalently binding the targeting ligand or other organic moiety to the components of the outer layer, various types of bonds and linking agents may be employed. Typical methods for forming such coupling include formation of amides with the use of carbodiamides, or formation of sulfide linkages through the use of unsaturated components such as maleimide. Other coupling agents include, for example, glutaraldehyde, propanedial or butanedial, 2-iminothiolane hydrochloride, bifunctional N-hydroxysuccinimide esters such as disuccinimidyl suberate, disuccinimidyl tartrate, bis[2-(succinimidooxycarbonyloxy)ethyl]sulfone, heterobifunctional reagents such as N-(5-azido-2-nitrobenzoyloxy)succinimide, succinimidyl 4-(N-maleimidomethyl)cyclohexane-1-carboxylate, and succinimidyl 4-(p-maleimidophenyl)butyrate, homobifunctional reagents such as 1,5-difluoro-2,4-dinitrobenzene, 4,4′-difluoro-3,3′-dinitrodiphenylsulfone, 4,4′-diisothiocyano-2,2′-disulfonic acid stilbene, p-phenylenediisothiocyanate, carbonylbis(L-methionine p-nitrophenyl ester), 4,4′-dithiobisphenylazide, erythritolbiscarbonate and bifunctional imidoesters such as dimethyl adipimidate hydrochloride, dimethyl suberimidate, dimethyl 3,3′-dithiobispropionimidate hydrochloride and the like. Linkage can also be accomplished by acylation, sulfonation, reductive amination, and the like. Commercially available linking systems include the HYNIC linker technology marketed by AnorMED, Langley, BC. A multiplicity of ways to couple, covalently, a desired ligand to one or more components of the outer layer is well known in the art.

For example, methods to effect direct binding are described in detail in U.S. Pat. No. 6,676,963, incorporated herein by reference; with respect to these methods.

The foregoing discussion is not comprehensive. In a specific case which employs aptamers, it may be advantageous to couple the aptamer to the nanoparticle by the use of a cationic surfactant as a coating to the particles.

The targeting agent itself may be any ligand which is specific for an intended target site. The target site will contain a “cognate” for the targeting agent or ligand—i.e., a moiety that specifically binds to the targeting agent or ligand. Familiar cognate pairs include antigen/antibody, receptor/ligand, biotin/avidin and the like. Commonly, such a ligand may comprise an antibody or portion thereof, an aptamer designed to bind the target in question, a known ligand for a specific receptor such as an opioid receptor binding ligand, a hormone known to target a particular receptor, a peptide mimetic and the like. Certain organs are known to comprise surface molecules which bind known ligands; even if a suitable ligand is unknown, antibodies can be raised and modified using standard techniques and aptamers can be designed for such binding.

Antibodies or fragments thereof are preferred targeting agents because of their capacity to be generated to virtually any target, regardless of whether the target has a known ligand to which it binds either natively or by design. Standard methods of raising antibodies, including the production of monoclonal antibodies are well known in the art and need not be repeated here. It is well known that the binding portions of the antibodies reside in the variable regions thereof, and thus fragments of antibodies which contain only variable regions, such as F_{ab}, Fᵥ, and scFᵥ moieties are included within the definition of “antibodies.” Recombinant production of antibodies and these fragments which are included in the definition are also well established. If the imaging is to be conducted on human subjects, it may be preferable to humanize any antibodies which serve as targeting ligands. Techniques for such humanization are also well known.

In addition to the targeting agent, the particles may further contain a biologically active agent, a labeling component or both. In some instances, this additional agent is unnecessary where the particles themselves inherently possess a desired property, such as the use of perfluorocarbon particles as contrast agents for ultrasound or MRI. Where the active composition inherently contains this “activity component,” it is preferred that the inactive carrier be comprised of vehicles that lack this property, however, because the vehicles in the inactive composition lack targeting agent, this is not an absolute requirement. For other applications, such as proton-based MRI, radionuclide imaging, and drug delivery, an additional “activity component” is desirable.

Typically, for most MRI contrast agents, the vehicles are coupled to a chelator in which a transition metal is disposed. Typical chelators include porphyrins, ethylenediaminetetraacetic acid (EDTA), diethylenetriamine-N,N,N′,N″,N″-pentaacetate (DTPA), 1,4,10,13-tetraoxa-7,16-diazacyclooctadecane-7 (ODDA), 16-diacetate, N-2-(azol-1(2)-yl)ethyliminodiacetic acids, 1,4,7,10-tetraazacyclododecane-N,N′,N″,N′″-tetraacetic acid (DOTA), 1,7,13-triaza-4,10,16-trioxacyclo-octadecane-N,N′,N″-triacetate (TTTA), tetraethylene glycols, 1,5,9-triazacyclododecane-N,N′,N″,-tris(methylenephosphonic acid) (DOTRP), N,N′,N″-trimethylammonium chloride (DOTMA) and analogues thereof.

Metal chelates for use in MRI imaging are well known. See, for example, U.S. Pat. Nos. 5,512,294 and 6,132,764 which describe liposomal particles with metal chelates; U.S. Pat. Nos. 5,064,636 and 5,120,527 which describe paramagnetic oil emulsions and U.S. Pat. Nos. 5,614,170 and 5,571,498 which describe emulsions that incorporate lipophilic gadolinium chelates as blood pool contrast agents. U.S. Pat. No. 5,804,164 describes water-soluble, lipophilic agents that contain chelating agents and paramagnetic metals. U.S. Pat. No. 6,010,682 describes lipid-soluble, chelated contrast agents that are administered in the form of liposomes, micelles or lipid emulsions.

Suitable paramagnetic metals include a lanthanide element of atomic numbers 58-70 or a transition metal of atomic numbers 21-29, 42 or 44, i.e., for example, scandium, titanium, vanadium, chromium, manganese, iron, cobalt, nickel, copper, molybdenum, ruthenium, cerium, praseodymium, neodymium, promethium, samarium, europium, gadolinium, terbium, dysprosium, holmium, erbium, thulium, and ytterbium, most preferably Gd(III), Mn(II), iron, europium and/or dysprosium.

The vehicles in the active composition may also be used for radionuclide imaging, radionuclides may be included by chelation in a manner similar to the metal ions complexed for use in MRI described above or alternative coupling mechanisms may be used. Radionuclides may be either therapeutic or diagnostic; diagnostic imaging using such nuclides is well known and by targeting radionuclides to undesired tissue a therapeutic benefit may be realized as well. Typical diagnostic radionuclides include ^{99m}Tc, ⁹⁵Tc, ¹¹¹In, ⁶²Cu, ⁶⁴Cu, ⁶⁷Ga, and ⁶⁸Ga, and therapeutic nuclides include ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, ¹⁹⁸Au, ¹⁶⁹ Yb, ¹⁷⁵Yb, ¹⁶⁵Dy, ¹⁶⁶Dy, ⁶⁷Cu, ¹⁰⁵Rh, ¹¹¹Ag, and ¹⁹²Ir.

The nuclide can be provided to a preformed emulsion in a variety of ways. For example, ⁹⁹Tc-pertechnate may be mixed with an excess of stannous chloride and incorporated into the preformed emulsion of nanoparticles. Stannous oxinate can be substituted for stannous chloride. In addition, commercially available kits, such as the HM-PAO (exametazine) kit marketed as Ceretek® by Nycomed Amersham can be used. Means to attach various radioligands to the nanoparticles of the invention are understood in the art. As stated above, the radionuclide may not be an ancillary material, but may instead occupy the chelating agent in lieu of the paramagnetic ion when the composition is to be used solely for diagnostic or therapeutic purposes based on the radionuclide.

In addition to or instead of a labeling component, the vehicles may contain a therapeutic agent. These biologically active agents can be of a wide variety, including proteins, nucleic acids, pharmaceuticals, radionuclides and the like. Thus, included among suitable pharmaceuticals are antineoplastic agents, hormones, analgesics, anesthetics, neuromuscular blockers, antimicrobials or antiparasitic agents, antiviral agents, interferons, antidiabetics, antihistamines, antitussives, anticoagulants, and the like.

As described above, while the biologically active agent may be passively included in the particles, the targeting agent or chelating agent is typically more firmly linked. Thus, in some cases, especially with respect to drugs, an “activity component” may be included in the surfactant layer if its properties are suitable. For example, if the component contains a highly lipophilic portion, it may simply be embedded in the lipid/surfactant coating. Further, if the component is capable of direct adsorption to the coating, this too will effect its coupling. For example, nucleic acids, because of their negative charge, adsorb directly to cationic surfactants.

In summary, in the methods of the invention, the active composition can be used for drug delivery, ultrasound imaging, X-ray imaging, radionuclide imaging, magnetic resonance imaging, tomography, or any other imaging technology dependent on signaling. In some instances, fluorescent labeling, using, for example, a fluorescent protein, fluorescein or dansyl is used.

The following examples are offered to illustrate but not to limit the invention.

### Preparation A

### Preparation of Lipid-Coupled Ligand Specific for αᵥβ₃

### Part A—DSPE-PEG(2000)Maleimide-Mercaptoacetic Acid Adduct

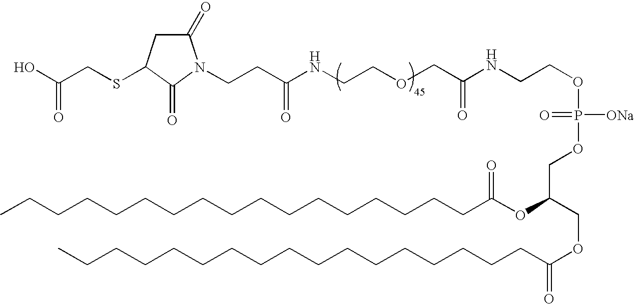

1,2-Distearoyl-sn-Glycero-3-Phosphoethanolamine-N-[Maleimide(Polyethylene Glycol)2000] is dissolved in DMF and degassed by sparging with nitrogen or argon. The oxygen-free solution is adjusted to pH 7-8 using DIEA, and treated with mercaptoacetic acid. Stirring is continued at ambient temperatures until analysis indicates complete consumption of starting materials. The solution is used directly in the following reaction.

### Part B—Conjugation of the DSPE-PEG(2000)Maleimide-Mercaptoacetic Acid Adduct With 2-[({4-[3-(N-{2-[(2R)-2-((2R)-2-Amino-3-sulfopropyl)-3-sulfopropyl]ethyl}carbamoyl)propoxy]-2,6-dimethylphenyl}sulfonyl)amino](2S)-3-({7-[(imidazol-2-ylamino)methyl]-1-methyl-4-oxo(3-hydroquinolyl)}carbonylamino)propanoic Acid

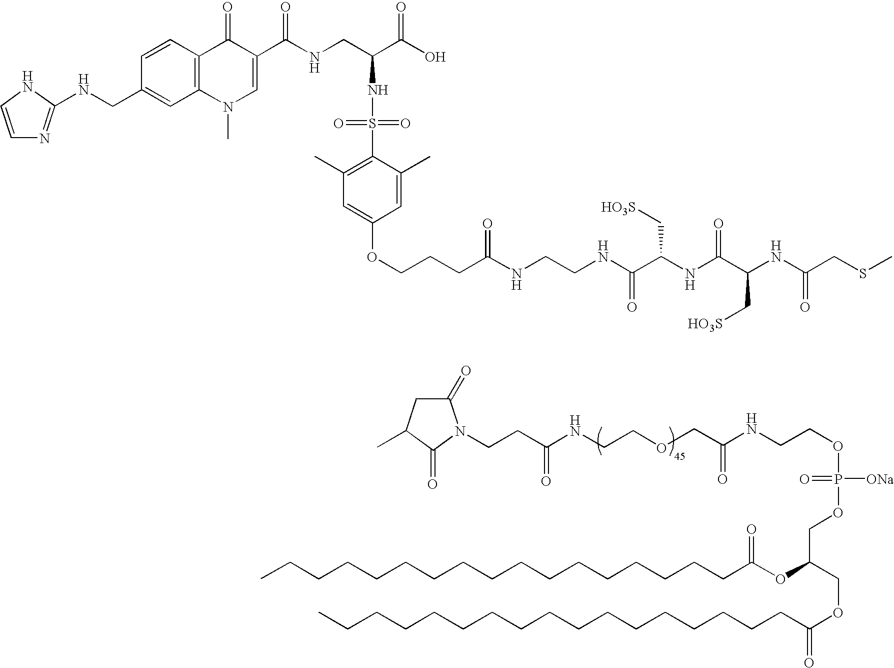

The product solution of Part A, above, is pre-activated by the addition of HBTU and sufficient DIEA to maintain pH 8-9. To the solution is added 2-[({4-[3-(N-{2-[(2R)-2-((2R)-2-amino-3-sulfopropyl)-3-sulfopropyl]ethyl}carbamoyl)propoxy]-2,6-dimethylphenyl}sulfonyl)amino](2S)-3-({7-[(imidazol-2-ylamino)methyl]-1-methyl-4-oxo(3-hydroquinolyl)}carbonylamino)propanoic acid, and the solution is stirred at room temperature under nitrogen for 18 h. DMF is removed in vacuo and the crude product is purified by preparative HPLC to obtain the Part B title compound.

### Preparation B

### Preparation of Nanoparticles

The nanoparticles were produced as described in Flacke, S., et al, Circulation (2001) 104:1280-1285. Briefly, the nanoparticulate emulsions were comprised of 40% (v/v) perfluorooctylbromide (PFOB), 2% (w/v) of a surfactant co-mixture, 1.7% (w/v) glycerin and water representing the balance.

The surfactant of control, i.e., non-targeted emulsions included 60 mole % lecithin (Avanti Polar Lipids, Inc., Alabaster, Ala.), 8 mole % cholesterol (Sigma Chemical Co., St. Louis, Mo.) and 2 mole % dipalmitoyl-phosphatidylethanolamine (DPPE) (Avanti Polar Lipids, Inc., Alabaster, Ala.).

αᵥβ₃-targeted paramagnetic nanoparticles were prepared as above with a surfactant co-mixture that included: 60 mole % lecithin, 0.05 mole % N-[{w-[4-(p-maleimidophenyl)-butanoyl]amino}poly(ethylene glycol)2000]1,2-distearoyl-sn-glycero-3-phosphoethanolamine (MPB-PEG-DSPE) covalently coupled to the αᵥβ₃-integrin peptidomimetic antagonist (Bristol-Myers Squibb Medical Imaging, Inc., North Billerica, Mass.), 8 mole % cholesterol, 30 mole % Gd-DTPA-BOA and 1.95 mole % DPPE.

The components for each nanoparticle formulation were emulsified in a M110S Microfluidics emulsifier (Microfluidics, Newton, Mass.) at 20,000 PSI for four minutes. The completed emulsions were placed in crimp-sealed vials and blanketed with nitrogen.

Particle sizes are determined at 37° C. with a laser light scattering submicron particle size analyzer (Malvern Instruments, Malvern, Worcestershire, UK) and the concentration of nanoparticles was calculated from the nominal particle size (i.e., particle volume of a sphere). Most of the particles have diameters less than 400 nm.

Perfluorocarbon concentration is determined with gas chromatography using flame ionization detection (Model 6890, Agilent Technologies, Inc., Wilmington, Del.). One ml of perfluorocarbon emulsion combined with 10% potassium hydroxide in ethanol and 2.0 ml of internal standard (0.1% octane in Freon®) is vigorously vortexed then continuously agitated on a shaker for 30 minutes. The lower extracted layer is filtered through a silica gel column and stored at 4-6° C. until analysis. Initial column temperature is 30° C. and is ramped upward at 10° C./min to 145° C.

### EXAMPLE 1

### Effect of Inactive Carrier Decoy

Nanoparticles were prepared using αᵥβ₃ as the targeting agent and ¹¹¹In as the labeling agent, essentially as described in Preparation B. The nanoparticles contained 10 copies of In per particle.

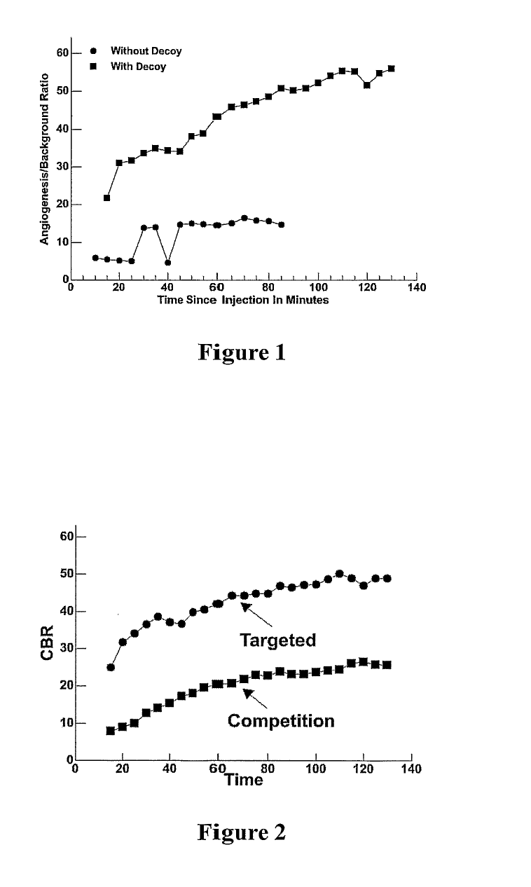

These particles were used as an active composition and administered a level of 0.5 mCi/kg to a rabbit bearing 18-day Vx-2 tumors. Imaging was performed with the Philips Genesys® system using a pinhole collimator. Significant targeting was seen by 15 minutes with good contrast. This procedure was repeated in the presence of a large excess of untargeted particles lacking any label. The contrast in the presence and absence of decoy is shown over a period of two hours in FIG. 1 which plots time after administration vs. the contrast—i.e., ratio of signal from target to background (CBR).

As shown, a significant improvement in contrast was achieved in the presence of decoy. Without the use of decoys, the persistence of the particles in circulation is inadequate to allow an efficacious amount of particles, i.e., radioactive signal, to accumulate at the target site. The co-administration of decoys extends the circulatory longevity of the indium-labeled particles which allows greater time for binding and signal enrichment at the target. To acquire the same level of signal at the target without decoys would require injecting far more, arguably unsafe levels, of indium-labeled particles to compensate for RES losses.

At higher doses of label, adjustments to the protocol may be needed to minimize nonspecific labeling.

### EXAMPLE 2

### Demonstration of Specific Binding

The results obtained in a similar experiment using particles that are targeted (not non-targeted) but unlabeled are shown in the analogous plot of FIG. 2, where the solid circles indicate the results when the targeted particles were administered alone and the solid squares represent the results when there was pre-administration of particles that are targeted, but not labeled. The competitive blockade of the labeled targeted composition administered with unlabeled targeted combination demonstrates the specificity of targeted composition for the pathology, rather than nonspecific accumulation. Thus, the specific binding enriches and maintains the effect, in this case a nuclear signal.

### EXAMPLE 3

### Effect of Increased Label Density on Vehicles

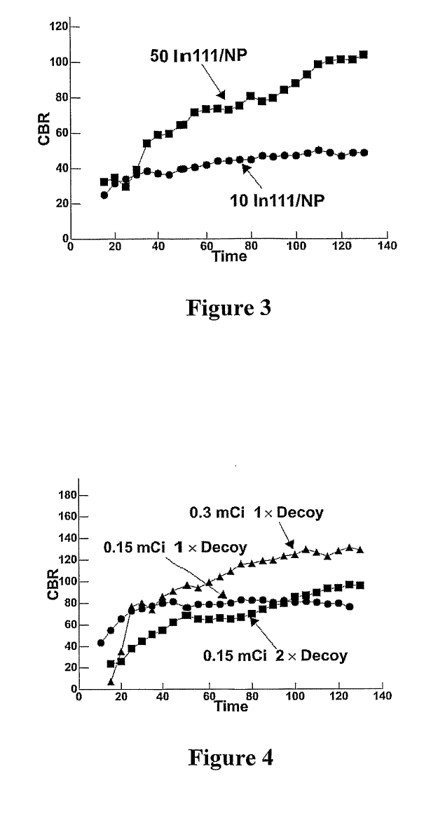

The experiment of Example 1 was repeated but comparing the results obtained when 0.3 mCi/kg were administered using labeled αᵥβ₃ targeted nanoparticles bearing 50 vs. 10 copies of In atoms per nanoparticle. As expected, as shown in FIG. 3, the more densely labeled particles provided enhanced contrast compound to background. As the number of radionuclides per particle increases, the potency of each bound particle to produce a detectable signal increases. Further, there is an increase in the ratio of decoys to radiolabeled particles. In this example, the ratio of decoys is increased 5-fold for the dosage using the 50 In/nanoparticle formulation vs. the 10/nanoparticle, since fewer active particles are needed, and the signal at the target site is not only improved per se, but enhanced for the same total dose of radioactivity.

### EXAMPLE 4

### Effect of Decoy Ratio

In the results shown in FIG. 4, indium labeled nanoparticles were administered in the presence of pre-measured amounts of emulsion containing comparable nanoparticles which lacked targeting agent and label. In the curve labeled 0.3 mCi, 1× decoy, sufficient particles to provide 0.3 mCi were administered along with 0.3 ml/kg of the decoy emulsion.

In the curve labeled 0.15 mCi, 1× decoy, the same amount of decoy emulsion was administered but the amount of labeled, targeted nanoparticles was reduced so that only 0.15 mCi were administered. As expected, as less label was administered, the resulting contrast was diminished even though the ratio of decoy to labeled active composition (targeted nanoparticles containing label) was doubled. However, if the amount of decoy emulsion was increased to 0.6 ml/kg, even though the level of label administered remained low at 0.15 mCi, the contrast signal was enhanced to mimic the curve obtained with 0.3 mCi. Thus, the results obtained shown in the curve labeled 0.15 mCi, 2× decoy, shows that at the same level of targeted nanoparticles, enhancing the ratio of decoy to active composition by two-fold improves the results.

1. A method to deliver a desired agent selectively to a target site in a subject, which method comprises administering, substantially simultaneously, to said subject:
(1) an active composition of targeted particulate vehicles wherein said vehicles are coupled to a ligand that binds specifically to its cognate at the target site and wherein said vehicles comprise, or are themselves, the agent to be delivered; and
(2) an inactive carrier which comprises particulate vehicles which lack said binding ligand and which optionally lack said agent;
wherein the ratio of vehicles in the carrier of (2) to vehicles in the composition of (1) is sufficient to enhance the association of the active composition with target site, and/or to lower the required dosage of targeted vehicles.

2. The method of claim 1 wherein the ratio of vehicles in the carrier (2) to vehicles in the composition of (1) is at least 1:1.

3. The method of claim 1 wherein the ratio of vehicles in the carrier (2) to vehicles in the composition of (1) is at least 100:1.

4. The method of claim 1 wherein the vehicles comprise liposomes, micelles, bubbles containing gas and/or gas precursors, lipoproteins, halocarbon and/or hydrocarbon nanoparticles, halocarbon and/or hydrocarbon emulsion droplets, hollow and/or porous particles and/or solid nanoparticles.

5. The method of claim 1 wherein the vehicles in the carrier of (2) and the vehicles in the composition of (1) are of the same composition.

6. The method of claim 1 wherein the vehicles in the carrier of (2) and the vehicles in the composition of (1) are not of the same composition.

7. The method of claim 1 wherein either the carrier of (2) or the composition of (1) or both is comprised of vehicles that are not of the same composition.

8. The method of claim 1 wherein at least one said agent is selected from the group consisting of a contrast agent for ultrasound; a magnetic resonance imaging (MRI) agent; a radionuclide; a therapeutic agent; and a fluorophore.

9. The method of claim 1 wherein the ligand that binds to cognate is an antibody or fragment thereof or is a peptidomimetic.

10. The method of claim 9 wherein said moiety targets αᵥβ₃.

11. A formulation which comprises
(1) an active composition of particulate targeted vehicles wherein said vehicles are coupled to a ligand that binds specifically to a cognate at a target site and wherein said vehicles comprise, or are themselves, an agent to be delivered to said target site; and
(2) an inactive carrier which comprises particulate vehicles which lack said binding ligand and which optionally lack said agent.

12. The formulation of claim 11 wherein the ratio of vehicles in the carrier of (2) to vehicles in the composition of (1) is sufficient to enhance the association of the active composition with the target site and/or to lower the required dosage of targeted vehicles.

13. The formulation of claim 11 wherein the ratio of vehicles in the carrier of (2) to vehicles in the composition of (1) is at least 1:1.

14. The formulation of claim 11 wherein the ratio of vehicles in the carrier of (2) to vehicles in the composition of (1) is at least 100:1.

15. The formulation of claim 11 wherein the vehicles comprise liposomes, micelles, bubbles containing gas and/or gas precursors, lipoproteins, halocarbon and/or hydrocarbon nanoparticles, halocarbon and/or hydrocarbon emulsion droplets, hollow and/or porous particles and/or solid nanoparticles.

16. The formulation of claim 11 wherein the vehicles in the carrier of (2) and the vehicles in the composition of (1) are of the same composition.

17. The formulation of claim 11 wherein the vehicles in the carrier of (2) and the vehicles in the composition of (1) are not of the same composition.

18. The formulation of claim 11 wherein either the carrier of (2) or the composition of (1) or both is comprised of vehicles that are not of the same composition.

19. The formulation of claim 11 wherein at least one said agent is selected from the group consisting of a contrast agent for ultrasound; a magnetic resonance imaging (MRI) agent; a radionuclide; a therapeutic agent; and a fluorophore

20. The formulation of claim 11 wherein the moiety that binds to cognate is an antibody or fragment thereof or is a peptidomimetic.

21. The formulation of claim 20 wherein said moiety targets αᵥβ₃.

22. A method to obtain an ultrasound image of a targetable site, which method comprises obtaining said image in a subject, wherein said subject has been administered, substantially simultaneously,
(1) an active composition of particulate targeted vehicles wherein the vehicles are coupled to a ligand that binds specifically to a cognate at the target site and comprise an ultrasound contrast agent, and
(2) an inactive carrier which comprises vehicles that lack said binding ligand,
in a ratio of vehicles in the carrier of 92) to vehicles in the active composition of (1) which is sufficient to enhance the association of the active composition with the target site, and/or to lower the required dosage of targeted vehicles.

23. The method of claim 22 wherein the vehicles in the active composition are perfluorocarbon nanoparticles and the vehicles in the inert carrier are oil droplets.

24. A method to obtain a proton magnetic resonance image of a target site in a subject which method comprises obtaining said image from a target in a subject that has been administered, substantially simultaneously,
(1) an active composition of particulate targeted vehicles wherein said vehicles are coupled to a ligand that binds specifically to a cognate at the target site and comprise a chelating agent containing a heavy metal ion; and
(2) an inactive carrier which comprises vehicles which lack said binding ligand and optionally lack said heavy metal ion,
wherein the ratio of vehicles in the carrier of (2) to the vehicles in the active composition of (1) is sufficient to enhance the association of the active composition with the target site, and/or to lower the required dosage of targeted vehicles.

25. The method of claim 24 wherein the chelating agent comprises DOTA and the binding moiety binds to αᵥβ₃.

26. A method to obtain an optical image of a target site, which method comprises obtaining said image in a subject, wherein said subject has been administered, substantially simultaneously,
(1) an active composition of particulate targeted vehicles wherein the vehicles are coupled to a ligand that binds specifically to a cognate at the target site and comprise a visible label, and
(2) an inactive carrier which comprises vehicles that lack said binding ligand,
in a ratio of vehicles in the carrier of (2) to vehicles in the active composition of (1) sufficient to enhance the association of the active composition with the target site, and/or to lower the required dosage of targeted vehicles.

27. The method of claim 26 wherein the visible label is fluorescent and the inactive carrier lacks said label.

28. A method to obtain an X-ray image of a target, which method comprises obtaining said image in a subject, wherein said subject has been administered, substantially simultaneously,
(1) an active composition of particulate targeted vehicles wherein the vehicles are coupled to a ligand that binds specifically to a cognate at the target site and comprise at least one X-ray opaque ligand, and
(2) an inactive carrier which comprises vehicles that lack said binding ligand,
in a ratio of vehicles in the carrier of (2) to vehicles in the active composition of (1) sufficient to enhance the association of the active composition with the target site, and/or to lower the required dosage of targeted vehicles.

29. The method of claim 28 wherein the vehicles in the inactive carrier lack said opaque moiety.

30. A method to obtain ¹⁹F magnetic resonance image of a target, which method comprises obtaining said image in a subject, wherein said subject has been administered, substantially simultaneously,
(1) an active composition of particulate targeted vehicles wherein the vehicles are coupled to a ligand that binds specifically to a cognate at the target site and comprise ¹⁹F, and
(2) an inactive carrier which comprises vehicles that lack said binding ligand,
in a ratio of vehicles in the carrier of (2) to vehicles in the active composition of (1) sufficient to enhance the association of the active composition with the target site, and/or to lower the required dosage of targeted vehicles.

31. The method of claim 30 wherein the vehicles in the active composition are perfluorocarbon nanoparticles and the vehicles in the inactive carrier are oil droplets.
